(19) United States
(12) Patent Application Publication
(10) Pub. No.: US 2006/0178668 A1
(43) Pub. Date: Aug. 10, 2006

(54) Electrode tip apparatus for electrosurgery with protective insulation arrangement

(76) Inventor: Ford D. Albritton IV, Dallas, TX (US)

Correspondence Address: DOCKET CLERK, P.O. DRAWER 800889, DALLAS, TX, 75380, US

(21) Appl. No.: 11/052,736

(22) Filed: Feb. 7, 2005

(51) Int. Cl. A61B 18/14

(52) U.S. Cl. 606/45; 606/49

(57) ABSTRACT

An electrode tip for electrosurgery is provided with a protective insulation arrangement that covers some, but not all, of the surgical end of the tip. This reduces the possibility of undesired harmful effects of electrical current delivered by the electrode tip during electrosurgery.

### FIELD OF THE INVENTION

The invention relates generally to electrosurgery and, more particularly, to electrode tips used in electrosurgery.

### BACKGROUND OF THE INVENTION

Electrosurgery refers to any process by which an electrode delivers AC electric current at a point of tissue contact to perform a surgical function. Examples of surgical procedures which can be accomplished using conventional electrosurgery techniques include anatomic dissection or hemostasis, and tissue removal through desiccation.

Of course, safety considerations arise whenever electrical power is used for any purpose, and electrosurgery is no exception. As in any situation where electrical power is utilized, it is possible that the electric current provided in the electrosurgery delivery electrode will produce undesired harmful effects

It is therefore desirable to reduce the possibility that the electric current delivered during electrosurgery will produce undesired harmful effects. Exemplary embodiments of the present invention provide on the surgical end of an electrode tip a protective insulation arrangement which covers some, but not all, of the surgical end. This permits electrosurgery to proceed, while also reducing the possibility that the electric current delivered during electrosurgery will produce undesired harmful effects.

### BRIEF DESCRIPTION OF THE DRAWINGS

FIG. 1 is a plan view of an electrode tip apparatus for electrosurgery according to exemplary embodiments of the invention.

FIG. 2 is a side view of the electrode tip apparatus of FIG. 1.

FIG. 3 is an isometric view of the electrode tip apparatus of FIG. 1.

FIG. 4 is a plan view of an electrode tip apparatus for electrosurgery according to further exemplary embodiments of the invention.

FIG. 5 is a side view of the electrode tip apparatus of FIG. 4.

FIG. 6 is an isometric view of the electrode tip apparatus of FIG. 4.

FIG. 7 is a plan view of an electrode tip apparatus for electrosurgery according to further exemplary embodiments of the invention.

FIG. 8 illustrates an electrosurgery apparatus according to exemplary embodiments of the invention.

FIG. 9 diagrammatically illustrates various examples of surface area covered by insulating material on the surgical end of an electrode tip apparatus according to exemplary embodiments of the invention.

FIG. 10 illustrates exemplary electrode tip apparatus manufacturing operations according to the invention.

### DETAILED DESCRIPTION

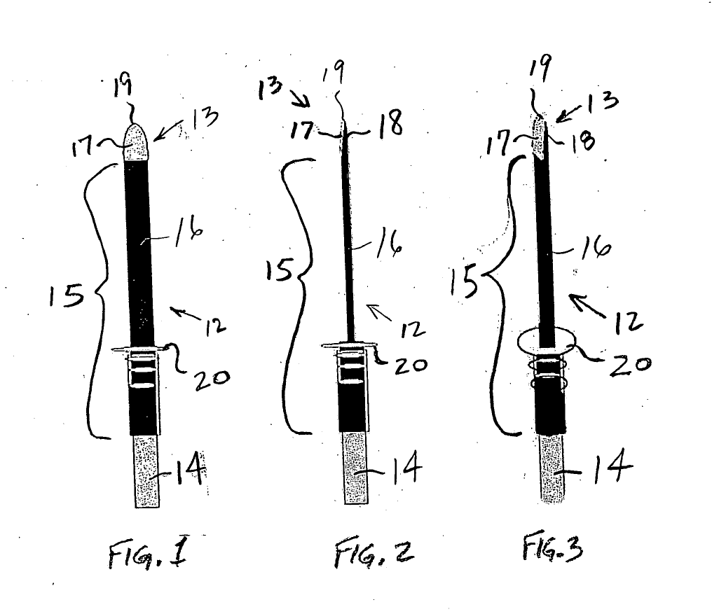

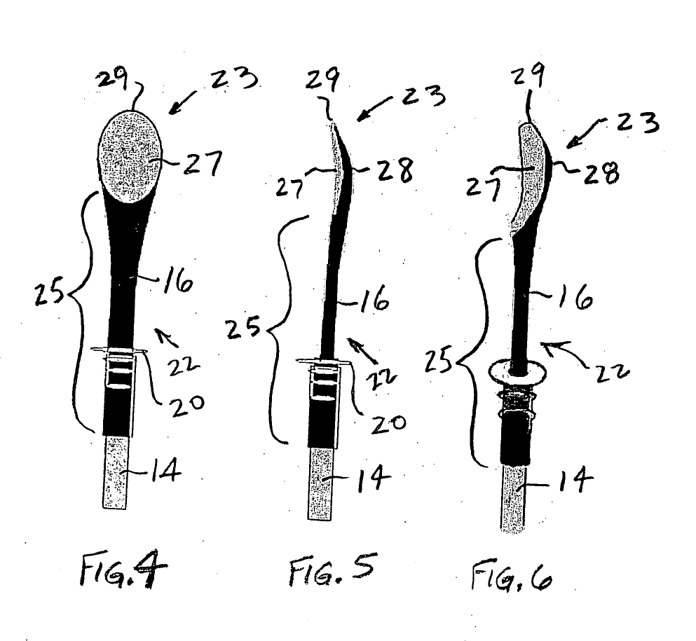

FIGS. 1, 2 and 3 are plan, side and isometric views, respectively, of an electrode tip apparatus for electrosurgery according to exemplary embodiments of the invention. The electrode tip apparatus of FIGS. 1-3 includes an electrically conductive electrode tip 12 with an insulating material 16 deposited thereon. The insulating material 16 is shown by dark shading. The electrode tip 12 includes a surgical end 13 which contacts the patient to perform electrosurgery. The electrode tip 12 further includes a connector end 14 for connecting to a source of electrical energy, and an elongated body 15 extending between the surgical end 13 and the connector end 14. An optional guard 20 is also carried on the elongated body 15.

In the exemplary embodiments of FIGS. 1-3, the insulating material 16 is deposited on the electrode tip 12 so as to cover the entire external surface area of the elongated body 15, and a portion of the external surface area of the surgical end 13. The conductive electrode tip 12 of FIGS. 1-3 is a straight “bovie” type tip as is well known in the art. The surgical end 13 basically defines two distinct surfaces, designated at 17 and 18, with an edge 19 therebetween. The surface portion 17, and possibly the edge 19, will contact the tissue of the patient during electrosurgery. The surface portion 18 is covered by the insulating material 16. The insulating material 16 can be electrically insulating, thermally insulating, or both electrically and thermally insulating. Accordingly, the insulating material 16 provided on the surface portion 18 of the surgical end 13 will function to insulate the surgical end 13 electrically, thermally, or both electrically and thermally, from patient tissue which is not subject to the surgical operation.

FIGS. 4, 5 and 6 are plan, side and isometric views, respectively, of an electrode tip apparatus according to further exemplary embodiments of the invention. The electrode tip apparatus of FIGS. 4-6 includes a curved spoon “bovie” type tip 22 with a curved, spoon-shaped surgical end 23. The electrode tip 22 further includes a connector end 14 as in FIGS. 1-3, and an elongated body 25 extending between the surgical end 23 and the connector end 14. In the exemplary embodiments of FIGS. 4-6, the insulating material 16 is deposited on and covers the elongated body 25 and a surface portion of the surgical end 23, designated at 28. The surgical end 23 basically defines two distinct surfaces, designated at 27 and 28, with an edge 29 therebetween. The surface portion 28 is generally convex in shape. The corresponding generally concave-shaped surface portion 27, and possibly the edge 29, will contact the patient to perform the surgical operation.

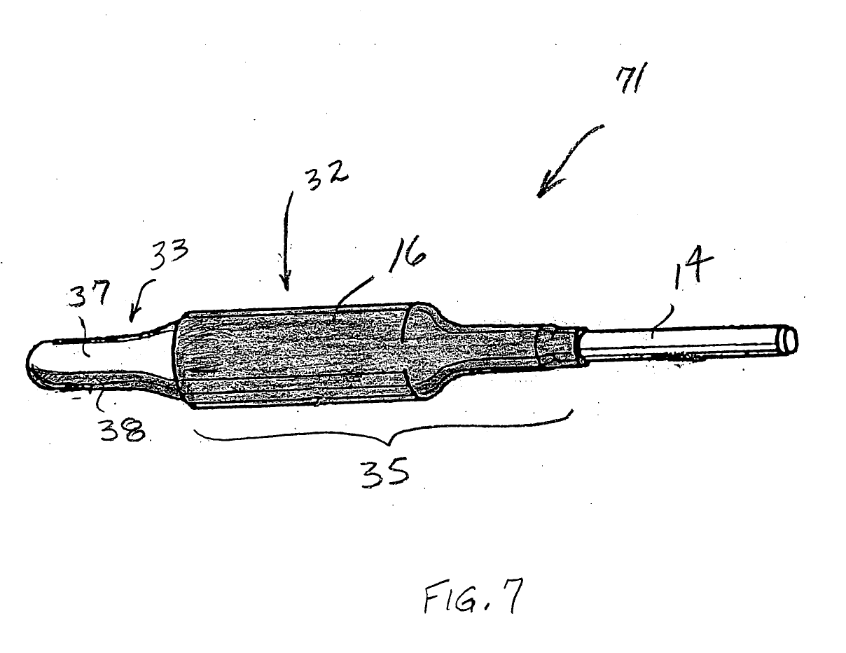

FIG. 7 is a plan view of an electrode tip apparatus 71 for electrosurgery according to further exemplary embodiments of the invention. The apparatus 71 includes an electrode tip 32 having a connector end 14, a surgical end 33 and an elongated body 35 extending therebetween. The insulating material 16 is deposited on the electrode tip so as to cover the elongated body 35 and a surface portion of the surgical end 33, designated at 38. Another surface portion of the surgical end 33, designated at 37, is not covered by the insulating material 16, and remains exposed for contacting the patient to perform the surgical operation. The surgical end 33 of FIG. 7 has a generally continuous external surface which includes the surface portions designated at 37 and 38.

The insulating material provided on each of the electrode tip apparatuses of FIGS. 1-7 can reduce collateral electrical and/or thermal energy transmission from the surgical end (13, 23, 33) of the electrode tip to patient tissue that is not involved in the surgical operation. This can reduce pain, avoid injury, and quicken healing.

The insulating material 16 of FIGS. 1-7 can be any suitable material which insulates electrically, thermally or both electrically and thermally. Some specific conventional examples of suitable insulating materials include polyphenylsulfone, polyarylethersulfone, polysulfone, polyethermide, and polycarbonate. Although the insulating material 16 covers the elongated body (15, 25, 35) of the electrode tip in the embodiments shown in FIGS. 1-7, coverage of the elongated body is not necessary to practice the invention. Other embodiments cover only a surface portion of the surgical end, without covering any of the elongated body. Still further embodiments cover a surface portion of the surgical end and only a portion of the elongated body.

In some embodiments, the electrode tips (12, 22, 32) of FIGS. 1-7 have a one-piece (unitary) construction. Other embodiments use a multi-component, composite construction.

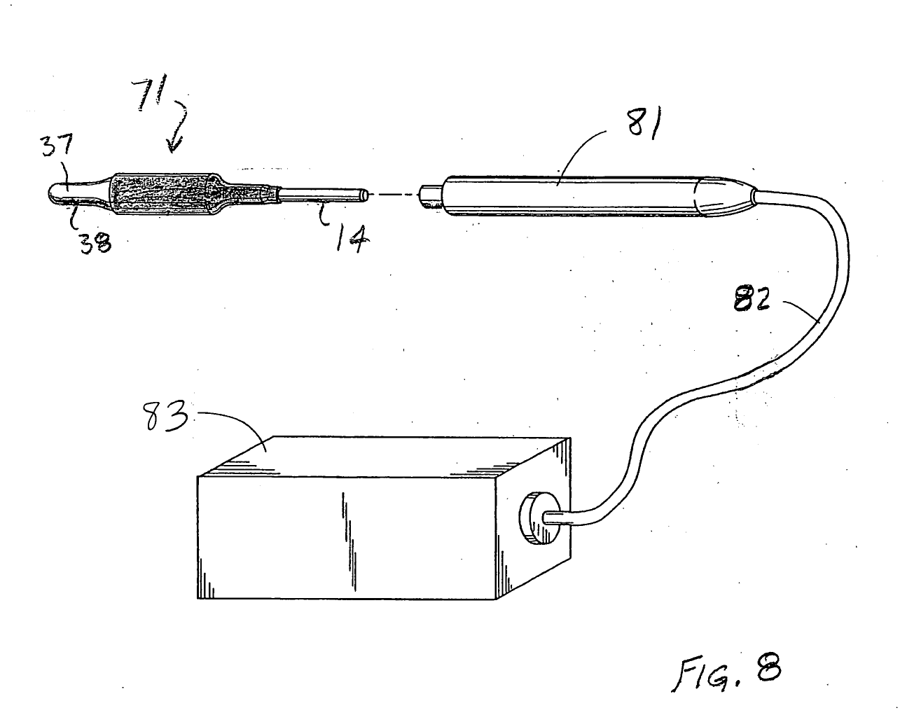

FIG. 8 illustrates an electrosurgery apparatus according to exemplary embodiments of the invention. The exemplary apparatus of FIG. 8 includes the electrode tip apparatus 71 of FIG. 7. As shown in FIG. 8, the connector end 14 of the electrode tip apparatus 71 is received into a handle 81 and is thereby removably fastenable to the handle 81. The handle 81 effectuates an electrical connection between the connector end 14 and a flexible electrically conductive cable 82. The cable 82 is connected, at an end thereof opposite the handle 81, to a power supply unit 83. The power supply unit 83 provides the electrode tip apparatus 71 with the current necessary for electrosurgery, via the cable 82 and the handle 81. In various exemplary embodiments, the cable 82 is removably connectable (e.g., pluggable) to either or both the handle 81 and the power supply unit 83.

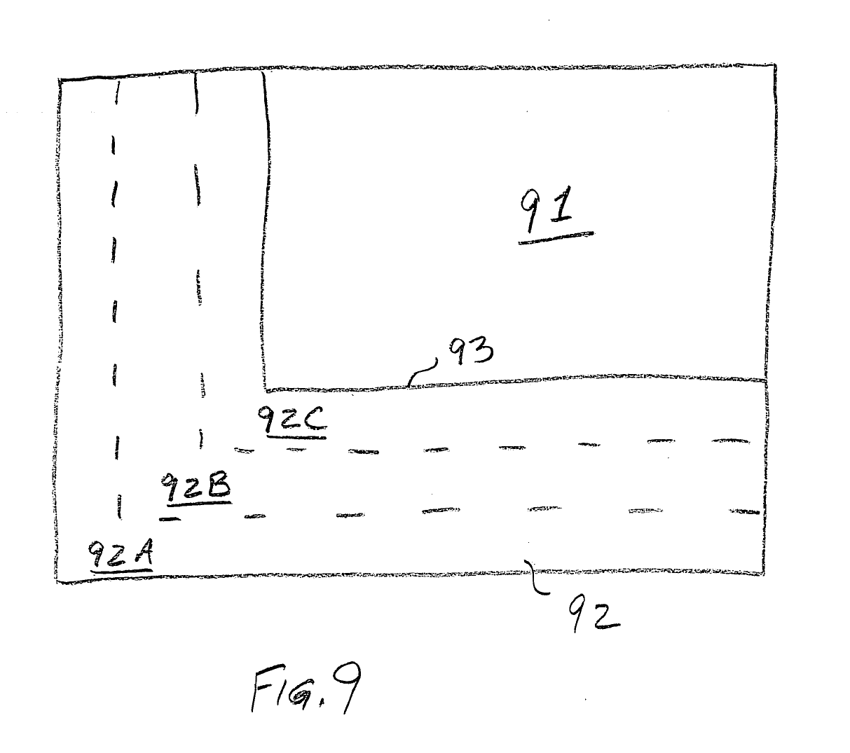

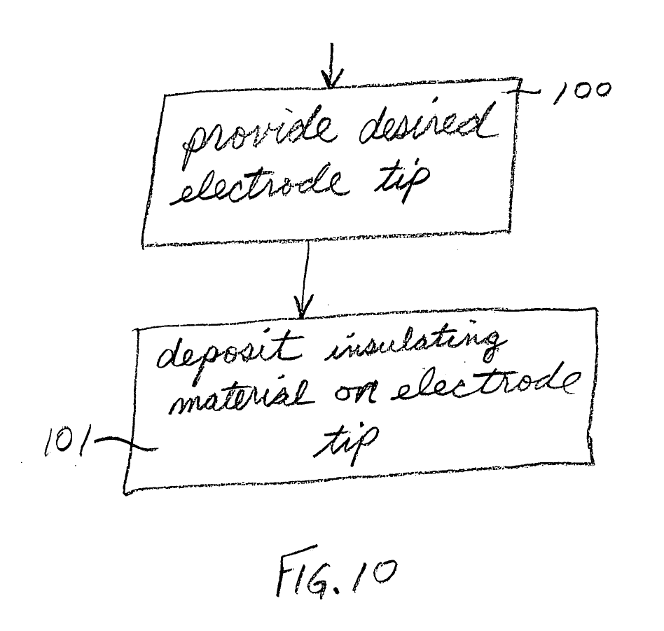

FIG. 9 diagrammatically illustrates various examples of insulating material coverage of the surface area of the surgical end of an electrode tip according to exemplary embodiments of the invention. FIG. 9 illustrates the concept of insulation material coverage on a surgical end. For clarity of exposition, the covered and uncovered surface portions of the surgical ends are depicted only from a conceptual standpoint, and FIG. 9 does not, of course, strictly reflect the actual shapes and relative sizes of the surface portions. Nevertheless, the surface portion representations of FIG. 9, together with the explanations provided hereinbelow, adequately convey various examples of insulating material coverage on the surgical ends.

The surface area that will contact the patient is represented generally at 91 in FIG. 9. The remaining surface area of the surgical end is represented at 92. The broken lines extending through the area 92 illustrate various examples of possible coverages of the insulating material on the surgical end of the electrode tip. In some embodiments, the entire surface portion illustrated at 92 can be covered by the insulating material. In other embodiments, only the area represented by 92A is covered by insulating material. In other embodiments, only the area represented by 92A and 92B is covered by insulating material. In other embodiments, only the area represented by 92A and 92C is covered with insulating material. In other embodiments, only the area represented by 92B and 92C is covered with insulating material. In other embodiments, only the area represented by 92B is covered with insulating material. In other embodiments, only the area represented by 92C is covered with insulating material. Furthermore, in any one of the embodiments described with respect to FIG. 9, the solid line 93 or either of the two broken lines can correspond to an edge between surface portions (see, e.g. 19 and 29 in FIGS. 1-6). As the size of the surface portion covered by insulating material increases, the level of patient protection increases, and the amount of current required for electrosurgery decreases. This latter factor can simplify the design of the power supply 83 in FIG. 8.

FIG. 10 illustrates exemplary operations that can be performed to manufacture an electrode tip apparatus (see, e.g., FIGS. 1-7) according to exemplary embodiments of the invention. After providing the desired electrode tip at 100, the desired insulating material (e.g., 16 in FIGS. 1-7) is deposited on the electrode tip at 101. The deposition at 101 can be performed according to any one of many well-known conventional deposition techniques.

Although exemplary embodiments of the invention have been described above in detail, this does not limit the scope of the invention, which can be practiced in a variety of embodiments.

What is claimed is:

1. An electrode tip apparatus for use in electrosurgery, comprising:
an electrode tip having a surgical end, a connector end for connecting to a source of electrical energy, and an elongated body extending between said surgical end and said connector end, said surgical end having a first surface portion which contacts a patient during electrosurgery, and said surgical end having a second surface portion; and
one of an electrical insulating material and a thermal insulating material deposited on said second surface portion of said surgical end, said insulating material covering said second surface portion without covering said first surface portion.

2. The apparatus of claim 1, wherein said insulating material is both an electrical insulating material and a thermal insulating material.

3. The apparatus of claim 1, wherein said first and second surface portions face in generally opposite directions.

4. The apparatus of claim 3, wherein said first surface portion is a generally concave surface portion and said second surface portion is a generally convex surface portion.

5. The apparatus of claim 1, wherein said surgical end defines a single continuous surface that includes said first and second surface portions.

6. The apparatus of claim 1, wherein said insulating material is interposed between said second surface portion and the patient when said first surface portion contacts the patient.

7. The apparatus of claim 1, wherein said second surface portion directly adjoins said first surface portion.

8. The apparatus of claim 1, wherein, when said first surface portion contacts the patient, said insulating material prevents the patient from contacting any other part of said electrode tip.

9. The apparatus of claim 1, wherein said insulating material prevents said second surface portion from contacting the patient when said first surface portion contacts the patient.

10. The apparatus of claim 1, wherein said elongated body has said insulating material deposited on a surface thereof.

11. The apparatus of claim 10, wherein said insulating material covers said elongated body completely.

12. The apparatus of claim 1, wherein said electrode tip has a unitary construction.

13. An electrosurgery apparatus, comprising:
an electrical power supply unit; and
an electrode tip assembly including an electrode tip, said electrode tip having a surgical end, a connector end electrically connected to said electrical power supply unit, and an elongated body extending between said surgical end and said connector end, said surgical end having a first surface portion which contacts a patient during electrosurgery, and said surgical end having a second surface portion, said electrode tip assembly further including one of an electrical insulating material and a thermal insulating material deposited on said second surface portion of said surgical end, said insulating material covering said second surface portion without covering said first surface portion.

14. The apparatus of claim 13, including a flexible electrical conductor connected to said electrical power supply unit, and a handle connected to said conductor, wherein said connector end of said electrode tip is removably fastened to said handle.

15. The apparatus of claim 13, wherein said electrode tip has a unitary construction.

16. A kit for assembling an electrosurgery apparatus, comprising:
an electrical power supply unit;
a handle;
a flexible electrical conductor for electrically connecting said electrical power supply unit to said handle; and
an electrode tip assembly including an electrode tip having a surgical end, a connector end, and an elongated body extending between said surgical end and said connector end, said surgical end having a first surface portion which contacts a patient during electrosurgery, and said surgical end having a second surface portion, said electrode tip assembly further including one of an electrical insulating material and a thermal insulating material deposited on said second surface portion of said surgical end, said insulating material covering said second surface portion without covering said first surface portion, and said connector end of said electrode tip removably fastenable to said handle.

17. The apparatus of claim 16, wherein said flexible electrical conductor is electrically connected to one of said electrical power supply unit and said handle.

18. The apparatus of claim 16, wherein said flexible electrical conductor is electrically connectable to one of said electrical power supply unit and said handle.

19. A method of manufacturing an electrode tip apparatus for use in electrosurgery, comprising:
providing an electrode tip having a surgical end, a connector end for connecting to a source of electrical energy, and an elongated body extending between said surgical end and said connector end; and
depositing one of an electrical insulating material and a thermal insulating material on a first surface portion of said surgical end to cover said first surface portion without covering a second surface portion of said surgical end that contacts a patient during electrosurgery.

20. The method of claim 19, wherein said depositing step includes depositing said insulating material on said elongated body.
